(19) United States
(12) Patent Application Publication
(10) Pub. No.: US 2025/0072821 A1
(43) Pub. Date: Mar. 6, 2025

(54) Sweat Extraction and Monitoring System

(71) Applicant: City University of Hong Kong, Hong Kong (CN)

(72) Inventors: Xinge YU, Hong Kong (CN); Guoqiang XU, Hong Kong (CN); Xingcan HUANG, Hong Kong (CN); Rui SHI, Hong Kong (CN); Yawen YANG, Hong Kong (CN); Pengcheng WU, Hong Kong (CN)

(21) Appl. No.: 18/457,426

(22) Filed: Aug. 29, 2023

(51) Int. Cl. A61B 5/00; A61B 5/145; A61B 10/00; H02N 1/04

(52) U.S. Cl. CPC A61B 5/4266; A61B 5/14521; A61B 5/14539; A61B 5/6833; A61B 10/0064; H02N 1/04

(57) ABSTRACT

A sweat extraction and monitoring system comprises a triboelectric nanogenerator (TENG) for inducing localized sweating for a human skin to generate sweat, iontophoresis electrodes electrically connected to the TENG and configured to be operably electrically contacting the human skin, at least one microfluidic channel for receiving the sweat, at least one biosensor for sensing the sweat received in the at least one microfluidic channel, and at least one sweat-activated battery configured to be actuatable by the sweat for powering the at least one biosensor.

### FIELD OF THE INVENTION

The present disclosure generally relates to sweat extraction and monitoring.

### BACKGROUND

The following discussion of the prior art is intended to present the invention in an appropriate technical context and allow its advantages to be properly appreciated. Unless clearly indicated to the contrary, however, reference to any prior art in this specification should not be construed as an express or implied admission that such art is widely known or forms part of common general knowledge in the field.

More and more attention has been paid to sweat extraction and monitoring in industries, such as in the fields of medical monitoring and clinical diagnostics. Non-invasive monitoring feature of wearable electronics with biosensors provides clinical relevant data yet user friendly formats. Sweat is an important body fluid, which is rich in biological and chemical substances or information, such as pH, inorganic ion concentrations and moisture, and thus can reflect nutritional and metabolic conditions. Various wearable sweat sensors have been developed and are used for real-time analysis of sweat biomarkers, including electrolytes, metabolites, and heavy metals. Despite the potential of sweat is an easy-to-get source for both on-demand and in situ analysis, its use and the way it is extracted are still limited. This is particularly an issue for sedentary humans, whose are less active and therefore it is difficult or ineffective to release sweat through natural movement, such as jogging.

It is an object of the present disclosure to overcome or substantially ameliorate one or more of the disadvantages of the prior art, or at least to provide a useful alternative.

### SUMMARY

In one aspect of the present disclosure there is provided a sweat extraction and monitoring system (SEMS). The SEMS comprise a triboelectric nanogenerator (TENG) for inducing localized sweating for a human skin to generate sweat, iontophoresis electrodes electrically connected to the TENG and configured to be operably electrically contacting the human skin, at least one microfluidic channel for receiving the sweat, at least one biosensor for sensing the sweat received in the at least one microfluidic channel, and at least one sweat-activated battery (SAB) configured to be actuatable by the sweat for powering the at least one biosensor.

In another aspect of the present disclosure there is provided a SEMS. The SEMS comprises a TENG for inducing localized sweating for a human skin to generate sweat, iontophoresis electrodes electrically connected to the TENG and configured to be operably electrically contacting the human skin, at least one microfluidic channel for receiving the sweat, a flexible electronic device, and at least one SAB. The flexible electronic device includes a flexible printed circuit board (FPCB), at least one biosensor disposed on the FPCB for sensing the sweat received in the at least one microfluidic channel to generate sensed data, and a microcontroller. The microcontroller is configured to transmit the sensed data to an external computer system. The at least one SAB is configured to be actuatable by the sweat for powering the at least one biosensor and the microcontroller.

In a further aspect of the present disclosure there is provided a self-powered wearable system. The self-powered wearable system comprises a wearable sweat apparatus and a computer system. The wearable sweat apparatus includes a TENG for inducing localized sweating for a human skin to generate sweat, iontophoresis electrodes electrically connected to the TENG and configured to be operably electrically contacting the human skin, at least one microfluidic channel for receiving the sweat, a flexible electronic device, at least one SAB, and a computer system. The flexible electronic device includes a FPCB, at least one biosensor disposed on the FPCB for sensing the sweat received in the at least one microfluidic channel to obtain sensed data, and a microcontroller electrically communicating with the at least one biosensor. The at least one SAB is configured to be actuatable by the sweat for powering the at least one biosensor and the microcontroller. The computer system is configured to electrically communicate with the microcontroller for receiving the sensed data.

Other example embodiments are discussed herein.

### BRIEF DESCRIPTION OF THE DRAWINGS

Embodiments of the present disclosure will now be described, by way of example only, with reference to the accompanying drawings in which:

FIG. 1 illustrates a SEMS according to certain embodiments of the present disclosure.

FIG. 2 illustrates a SEMS according to certain embodiments of the present disclosure.

FIG. 3A illustrates a SEMS for sweat extraction from a human skin and various components of the SEMS according to certain embodiments of the present disclosure.

FIG. 3B illustrates a TENG-based approach to stimulate sweating for the SEMS of FIG. 3A.

FIG. 3C shows optical images of the SEMS of FIG. 3A: (I) physical images of the microcircuit with biosensors; (II) remarkable bending ability of FPCB; (III) encapsulated TENG and microcircuit in polydimethylsiloxane (PDMS); and (IV) secure fit of the SEMS on the skin surface.

FIG. 3D illustrates the operation flowchart of the SEMS of FIG. 3A.

FIG. 3E illustrates a specific structure for iontophoresis electrodes of the SEMS according to certain embodiments of the present disclosure, where a hydrogel patch is formed on the iontophoresis electrodes.

FIG. 3F illustrates a TENG with a layered structure according to certain embodiments of the present disclosure.

FIG. 4A illustrates TENG's voltage output under different pressures according to certain embodiments of the present disclosure.

FIG. 4B illustrates TENG's current output under different pressures according to certain embodiments of the present disclosure.

FIG. 4C illustrates TENG's peak power under different pressures according to certain embodiments of the present disclosure.

FIG. 4D illustrates the stimulated sweat rate under different forces according to certain embodiments of the present disclosure.

FIG. 4E illustrates the stimulated sweat rate under different carbachol concentration when the TENG operates at a frequency of 2 Hz according to certain embodiments of the present disclosure.

FIG. 4F illustrates the stimulated sweat extraction rate under different pressures and carbachol concentrations according to certain embodiments of the present disclosure.

FIG. 4G illustrates a device for testing of the sweat flow (left) and optical image of microfluidic channels (right) according to certain embodiments of the present disclosure.

FIG. 4H illustrates numerically simulated concentration distributions in the microfluidic channels at different time under different number of the inlets (flow rate 1.5 μl/min) according to certain embodiments of the present disclosure.

FIG. 4I illustrates numerically simulation of the concentration distributions in the microfluidic channels at different time instances (inlet flow rate, 1.5 μL/min) according to certain embodiments of the present disclosure, where the number of inlets is seven.

FIG. 4J shows photograph of microfluidic channels by injecting colour-marked liquid under the test according to certain embodiments of the present disclosure.

FIG. 5A shows comparison of SABs when the anode is provided with or without silver oxide according to certain embodiments of the present disclosure.

FIG. 5B shows short-circuit current of SABs with different electrodes distance according to certain embodiments of the present disclosure.

FIG. 5C shows short-circuit current of SABs with three different thickness of magnesium (Mg) foil according to certain embodiments of the present disclosure.

FIG. 5D shows current density and power density curves of SABs according to certain embodiments of the present disclosure.

FIG. 5E shows open-circuit voltage of SABs as a function of time according to certain embodiments of the present disclosure.

FIG. 5F shows the capacity of SABs according to certain embodiments of the present disclosure.

FIG. 5G shows the electrical response of SABs as a function of time with increasing injected sweat volume according to certain embodiments of the present disclosure.

FIG. 5H shows the electrical output response of SABs under continuous bending and twisting according to certain embodiments of the present disclosure.

FIG. 5I shows the electrical output response of SABs bended as 45°, 90°, and 180° as a function of time according to certain embodiments of the present disclosure.

FIG. 6A shows the voltage vs time relationship of the Na+ sensor according to certain embodiments of the present disclosure.

FIG. 6B shows the voltage vs concentration relationship of the Na+ sensor according to certain embodiments of the present disclosure.

FIG. 6C shows the anti-interference characterization of the Na+ sensor according to certain embodiments of the present disclosure.

FIG. 6D shows the voltage vs time relationship of the pH sensor according to certain embodiments of the present disclosure.

FIG. 6E shows the voltage vs pH relationship of the pH sensor according to certain embodiments of the present disclosure.

FIG. 6F shows the anti-interference characterization of the pH sensor according to certain embodiments of the present disclosure.

FIG. 6G shows the voltage vs time relationship of the K+ sensor according to certain embodiments of the present disclosure.

FIG. 6H shows the voltage vs concentration relationship of the K+ sensor according to certain embodiments of the present disclosure.

FIG. 6I shows the anti-interference characterization of the K+ sensor according to certain embodiments of the present disclosure.

FIG. 7 illustrates a self-powered wearable system according to certain embodiments of the present disclosure.

FIG. 8A illustrates the versatility of wearable TENG devices and energy collection from different parts of the human body according to certain embodiments of the present disclosure.

FIG. 8B illustrates collection of sweat information through localized movement with a TENG device according to certain embodiments of the present disclosure.

FIG. 8C illustrates obtaining sweat information through NFC functionality with a TENG device according to certain embodiments of the present disclosure.

FIG. 8D illustrates the effects of TENG stimulation on pH concentration, Na+ concentration, and K+ concentration according to certain embodiments of the present disclosure.

FIG. 8E illustrates the pH values of sweat information from different body locations according to certain embodiments of the present disclosure.

FIG. 8F illustrates the K+ concentration of sweat information from different body locations according to certain embodiments of the present disclosure.

FIG. 8G illustrates the Na+ concentration of sweat information from different body locations according to certain embodiments of the present disclosure.

### DETAILED DESCRIPTION

The present disclosure will now be described with reference to the following examples which should be considered in all respects as illustrative and non-restrictive. In the Figures, corresponding features within the same embodiment or common to different embodiments have been given the same or similar reference numerals.

Throughout the description and the claims, the words “comprise”, “comprising”, and the like are to be construed in an inclusive sense as opposed to an exclusive or exhaustive sense; that is to say, in the sense of “including, but not limited to”.

Example embodiments relate to systems and methods that improve sweat extraction and monitoring for humans, thereby bettering the measurement, detection, and/or monitoring of humans' health conditions.

Many existing systems and methods stimulate or induce sweat from a human body by natural movement carried out by the human, such as jogging or waving arms. These systems and methods have many disadvantages. For example, their applications may be limited as monitoring of health conditions of the human body through analyzing the sweat can only occur when the person is taking sufficient exercise or movement to generate sweat. Further, they are not applicable to sedentary humans who do not like outdoor activities or who need to stay indoor for most of their weekdays. Moreover, how much time the sweat can be extracted as well as the amount of the extracted sweat are hard to control as it may depend on the person's physical conditions and the amount of exercise. Thus, these systems and methods are undesirable when efficiency is of a great consideration. Some existing systems introduce one or more external power supplies (such as one or more batteries) to generate electrical stimulation for sweat extraction. As the electric current is typically in the order of mA·cm⁻², these external power supplies may cause burden for thin, soft and skin-interfaced electronics.

Example embodiments solve one or more of these problems associated with the existing systems and methods by providing technical solutions that are unconventional, satisfactory, and favourable for scenarios in which one or more advantages of embodiments of the present disclosure are preferred.

One or more embodiments recognize the benefits of iontophoresis in assisting in sweat extraction. With iontophoresis, electric current is applied through the human skin for ionizing or charging molecules to allow their transportation across the stratum corneum. The advantages of employment of iontophoresis include being a non-invasive, needle-free process for extracting sweat from the human skin. Therefore, one or more embodiments combine self-powering technologies and iontophoresis to realize passive sweat collection without additional powering burden.

One or more embodiments include a triboelectric nanogenerator (TENG). The TENG is used for conversion of mechanical energy to electrical energy based on the synergistic effect of contact electrification and electrostatic induction. The TENG provides various advantages, such as low cost, a wide range of material sources to be selected, light weight, high efficiency at low operating frequencies, etc.

One or more embodiments use a TENG for self-powered iontophoresis-based sweat collection, and realize a fully self-powered sweat extraction, sensing, and wireless data transmission based on the iontophoresis. The TENG-based sweat collection module allows extraction of sweat from humans without extensive activities, which is particularly helpful for sedentary individuals, and accumulation of the extracted sweat in one or more microfluidic channels. The accumulated sweat can then be used to actuate or trigger or energize at least one sweat-activated battery (SAB). In the meanwhile, biological information of the humans can be obtained by analyzing the extracted sweat, such as through one or more biosensors.

According to one or more embodiments, advanced mechanics and materials designs are provided, which not only allow the self-powered sweat collection module to effectively extract and accumulate sweat in the microfluidic channels but also rapidly activate the SAB to provide power for a microcontroller or a microcontroller unit (MCU) and biosensors. This dispenses with the necessity of an external power source to power various electronics of the system.

One or more embodiments include a system with a dual-mode operation based on the TENG and SAB(s). This can be particularly helpful for sedentary people to conquer the difficulty-to-sweat challenges in these groups. The biological information in sweat can be detected by analysing the TENG-stimulated sweat. In addition, the skin-interfaced SAB with high output power density can be triggered by the TENG-induced sweat, and used to drive electronics and biosensors. According to one or more embodiments, the pH value, the sodium ion (Na+) concentration, and the potassium ion (K+) concentration in sweat can be sensed by the biosensors and the obtained or sensed data can be transmitted to a computer system wirelessly, such as through a near field communication (NFC) function.

One or more embodiments include a system that is particularly suitable for sedentary people. By tapping the TENG-based device or system, the sedentary people can passively extract sweat based on the iontophoresis process to detect biological information in sweat. The device may have flexible wearability characteristics, enabling real-time and non-invasive detection of sweat analytes.

One or more embodiments include a system that has unique advantages, including the ability to extract and analyse large quantities of sweat, which could provide accurate and detailed information about the body's hydration status and metabolic activity.

One or more embodiments provide experimental studies for one or more systems of certain embodiments on sedentary individuals. These experiments demonstrate the accuracy of the sweat sensors and the high feasibility of using the self-powered sweat extraction technology with self-powered sweat sensors according to certain embodiments for continuous healthcare monitoring of people, such as the elderly.

According to one or more embodiments, the system can be used for self-powered sweat collection, sensing sweat analytes and data transmission, and therefore for continuous intelligent healthcare monitoring. The system has significant potential in various applications in industries, including sports medicine, fitness, healthcare, etc.

According to one or more embodiments, in sports medicine, the system may be used by athletic trainers and medical professionals to measure the sweat rate and composition of athletes during training and competition. This information can help identify early signs of dehydration and electrolyte imbalances, thereby allowing for effective hydration strategies and potentially reducing the risk of heat-related illnesses.

According to one or more embodiments, in the fitness industry, the system may be used to monitor the hydration status and workout intensity of gym-goers, thereby potentially improving the effectiveness of exercise programs and reducing the risk of injury.

According to one or more embodiments, in the healthcare industry, the system may be used to diagnose certain medical conditions, such as cystic fibrosis that can affect sweat gland function. The system may also be used to monitor the sweat rate and composition of patients with chronic diseases, such as diabetes, to help manage their symptoms.

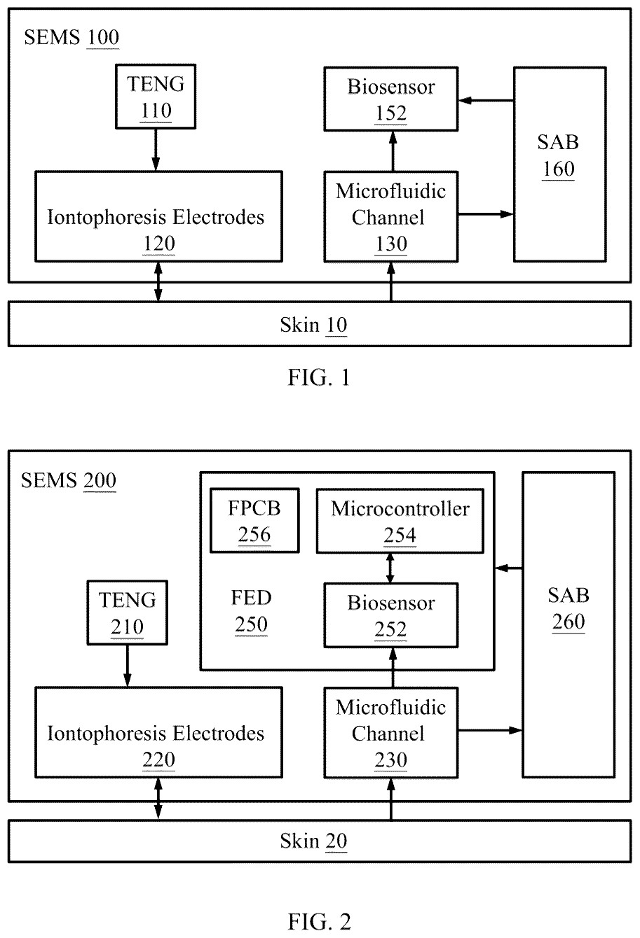

FIG. 1 illustrates a sweat extraction and monitoring system (SEMS) 100 according to certain embodiments. As illustrated, the SEMS 100 comprises a TENG 110, iontophoresis electrodes 120, at least one microfluidic channel 130, at least one biosensor 152, and at least one SAB 160.

The TENG 110 stimulates, induces, or activates localized sweating for a human skin 10 such that sweat can be generated desirably for use, such as for testing or analyzing to obtain health-related information for humans. The iontophoresis electrodes 120 are electrically connected to the TENG 110 and operably electrically contact the human skin 10 such that a closed circuit is formed. The stimulated sweat can flow into and received within the microfluidic channel 130. The biosensor 152 senses or detects or measures the sweat received in the microfluidic channel 130 such that biological information is obtained. Some of the stimulated sweat flowing within the microfluidic channel 130 triggers the SAB 160 such that the SAB 160 provides electrical power to the biosensor 152, thereby dispensing with any external power source. As such, the system 100 is a self-powered system, without any external battery to provide power.

In some embodiments, the iontophoresis electrodes 120 are loaded with carbachol. Carbachol can facilitate generation of sweat, such as promoting rapid sweating. As a result, the sweat extraction can be expedited. This is advantageous for applications where a rapid test is desirable or a large number of individuals are waiting for testing.

FIG. 2 illustrates a SEMS 200 according to certain embodiments. The SEMS 200 comprises a TENG 210 for inducing localized sweating for a human skin 20 to generate sweat, iontophoresis electrodes 220 electrically connected to the TENG 210 and configured to be operably electrically contacting the human skin 20, at least one microfluidic channel 230 for receiving the sweat, a flexible electronic device (FED) 250 including a flexible printed circuit board (FPCB) 256, at least one biosensor 252 disposed on the FPCB 256 for sensing the sweat received in the at least one microfluidic channel 230, and a microcontroller 254, the microcontroller 254 being configured to transmit the sensed data to an external computer system, and at least one SAB 260 is configured to be actuatable by the sweat for powering the at least one biosensor 252 and the microcontroller 254. In some embodiments, the iontophoresis electrodes 220 are carbachol-loaded.

The external computer system is an electrical device or system with computing capacity. It may be a computer server, a tablet, a laptop, an iPad, a smartphone, etc. that can receive data from or exchange data with the microcontroller 254 via one or more networks. The microcontroller 254 may receive and store the data sensed by the biosensor 252, and forward the sensed data to the external computer system via one or more network ports. The microcontroller 254 may process the received data and/or generate instructions to control operation of the SEMS 200. The microcontroller 254 may receive instructions from the external computer system for controlling operation of the SEMS 200.

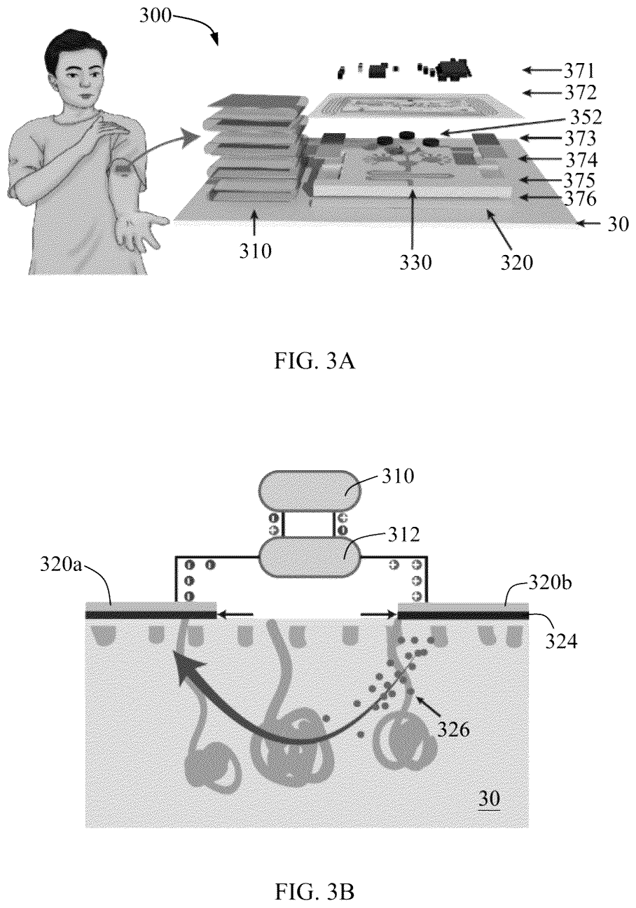

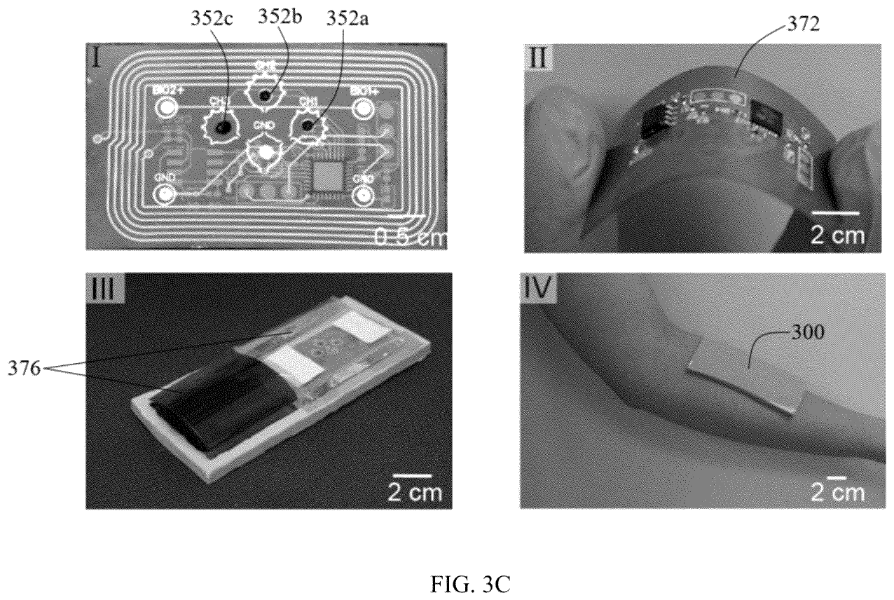

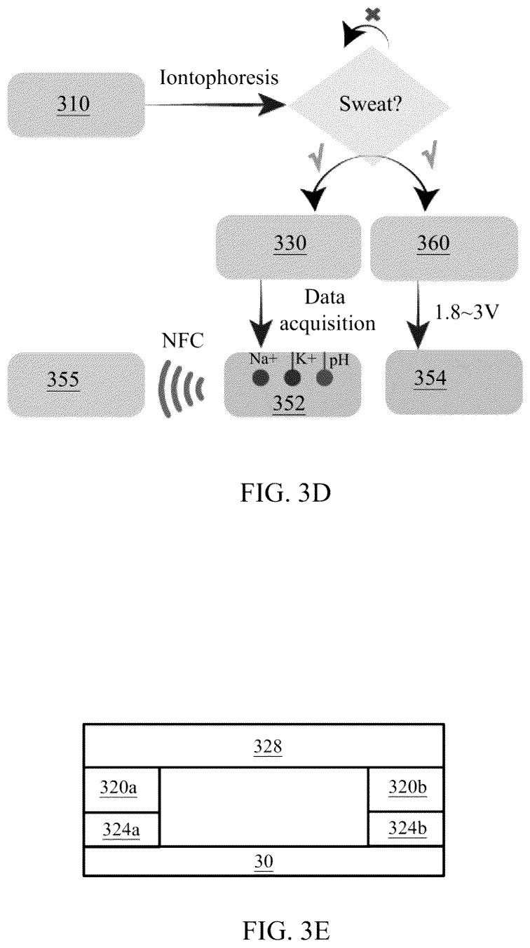

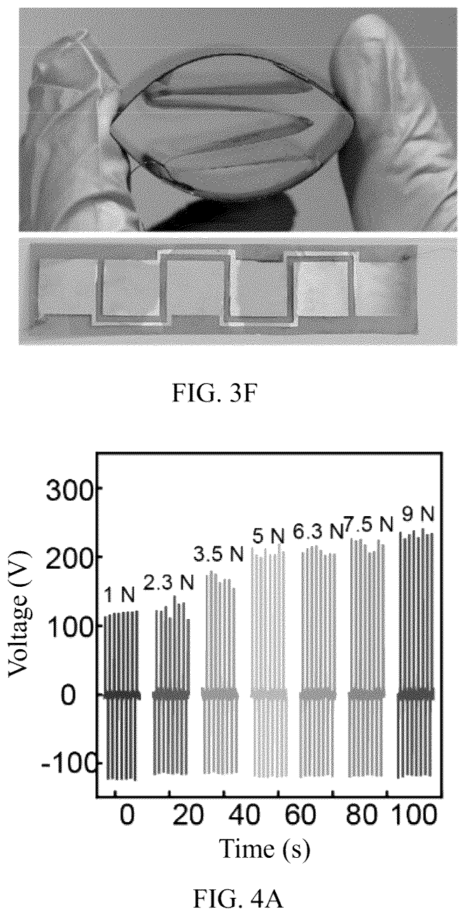

Referring to FIGS. 3A, 3B, 3C, 3D, 3E, and 3F, a SEMS 300 contacts a human skin 30 for sweat stimulation. The human skin 30 may be a skin of a certain part of the human body, such as arm, leg, foot, etc. The human skin 30 may be the skin of an on-demand sedentary person. The SEMS 300 can collect sweat from the human skin 30 for purpose of analyzing the sweat, such that the health conditions of the tested subject can be obtained or monitored. The SEMS 300 may be encapsulated with polydimethylsiloxane (PDMS) 376 to ensure a secure fit on the surface of the human skin 30 (FIG. 3C).

FIG. 3A illustrates a TENG 310, iontophoresis electrodes 320, one or more microfluidic channels 330, one or more biosensors 352, electronic elements 371, a flexible printed circuit board (FPCB) 372, magnesium (Mg) 373, silver oxide-deposited carbon cloth (Ag₂O/carbon cloth) 374, absorbent paper 375, and PDMS 376.

The SEMS 300 includes a TENG-based module. The TENG-based module includes the TENG 310 and the iontophoresis electrodes 320. The iontophoresis electrodes 320 has an anode 320a and a cathode 320b. A rectifier 312 bridges the TENG 310 and the iontophoresis electrodes 320 (FIG. 3B). In operation, the rectifier 312 may convert the alternating current (AC) generated by the TENG 310 to direct current (DC). This is advantageous as it can increase the effective power for stimulating sweat, thereby improving the efficiency for sweat extraction.

In some embodiments, the electrodes 320a and 320b may be formed (such as printed) on a flexible substrate, such as a flexible polyimide substrate 328. Further, a hydrogel patch may be disposed on the electrodes 320a and/or 320b. In the present embodiment as illustrated in FIG. 3E, the electrode 320a is an anode and the electrode 320b is a cathode. A sodium chloride (NaCl)-loaded hydrogel patch 324a (first hydrogel patch) is disposed on the electrode 320a, and a carbachol-loaded hydrogel patch 324b (second hydrogel patch) is disposed on the electrode 320b. In operation, the hydrogel patches 324a and 324b directly contact the human skin 30. Provision of the hydrogel patches 324a and 324b ensures stable or improved contact for the electrodes 320a and 320b with the human skin 30, which benefits the sweat extraction. In some other embodiments, one or both of the hydrogel patches may be loaded with other proper materials, or may be not loaded with any materials.

In the embodiment referring to FIG. 3B, the iontophoresis electrodes 320 are loaded with carbachol 326. When the TENG 310 is tapped, the carbachol 326 is delivered to the skin 30 through an iontophoresis process, thereby facilitating or inducing localized sweating of the skin 30. The generated sweat is then transported through the microfluidic channels 330 to the biosensors 352 such that the sweat can be sensed or detected for obtaining biological information reflecting one or more aspects of the health conditions.

At least one SAB 360 may be placed on either side of the microfluidic channels 330. The extracted sweat from the skin 30 can actuate or trigger the SAB 360. Specifically, the sweat acts as electrolyte. Absent the sweat, there is no electrical connection between the positive and negative electrodes of the SAB 360, and therefore no chemical reaction occurs. When the extracted sweat flows to and communicates the SAB 360, it serves as electrolyte and electrically connects the positive and negative electrodes and completes the circuit, thereby enabling chemical reactions. As a result, the SAB 360 can function as a power source and provide electricity sufficient to power various electronics, such as the biosensors 352 and the microcontroller 354 for data acquisition, processing, and/or storage of multiplexed monitoring of metabolic biomarkers in sweat. The data may be transmitted to a user interface 355 via NFC for reviewing (FIG. 3D).

The one or more biosensors 352 may be disposed properly. In the present embodiments, three biosensors 352 are positioned on three electrodes 352a, 352b, and 352c respectively that are attached to the FPCB 372 (FIG. 3C). One of the three electrodes 352a, 352b, and 352c may serve as a reference electrode. The remarkable flexibility of the FPCB 372 allows for customization according to specific required design.

By way of example, the three biosensors may include a Na+ sensor, a K+ sensor, and a pH sensor for sensing the Na+ concentration, the K+ concentration, and the pH values respectively. The Na+ sensor may include a layer of poly(3,4-ethylenedioxythiophene: poly(sodium 4-styrenesulfonate) (PEDOT: PSS) and a layer of ionophore disposed onto the layer of PEDOT: PSS and serving as a sensing area for sensing the sodium ions. The PEDOT layer acts as a transducer, while the layer of ionophore functions to bind ions. Similarly, the K+ sensor may include a layer of PEDOT: PSS and a layer of ionophore disposed onto the layer of PEDOT: PSS and serving as a sensing area for sensing the potassium ions. The pH sensor may include polyaniline (PANI).

By way of example, various microelectronic components or electronic elements 371 may be provided to enable or facilitate processing of data sensed by the biosensors 352. The sensed data may be transmitted to an external computer system via a wired protocol, or wireless protocol, such as Bluetooth, NFC, ultra-wide band (UWB), Zigbee, or WiFi.

Referring again to FIG. 3B, operation of the TENG 310 is a self-powered electrophoretic process. When the TENG 310 is tapped, it creates an electrical field that penetrates skin for sweat activation. To improve or maximize the power output of the TENG 310, the TENG 310 can be formed as a stacked layer structure (FIG. 3F). The stacked layer structure may include a plurality of polyimide layers folded from a polyimide film, copper electrodes formed on each of the plurality of polyimide layers, and fluorinated ethylene propylene (FEP) configured to be alternatively attached (such as coated) onto the copper electrodes to form FEP-attached copper electrodes. The copper electrodes that are not attached with FEP and the FEP-attached copper electrodes constitute output terminals of the TENG 310. In some embodiments, a thin layer of gold (Au, 35 mm×35 mm, thickness of 100 nm) film is deposited on a polyimide (PI, 25 μm) film, and a FEP film is provided partially covering the electrode and serves as the triboelectrification layer.

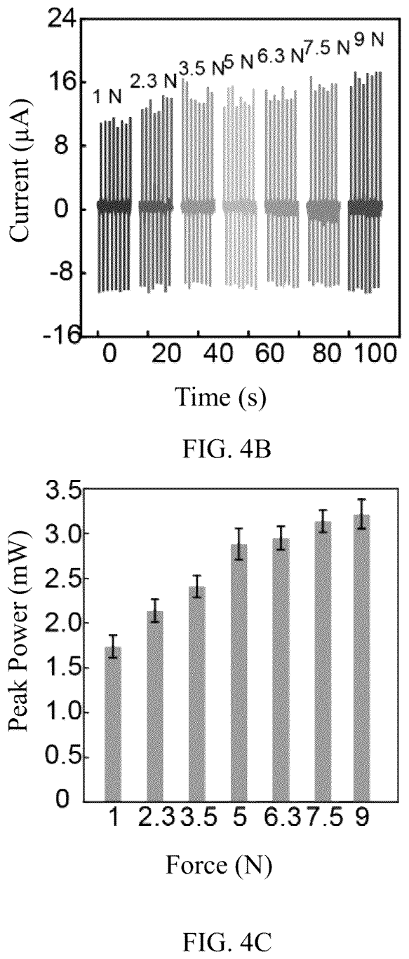

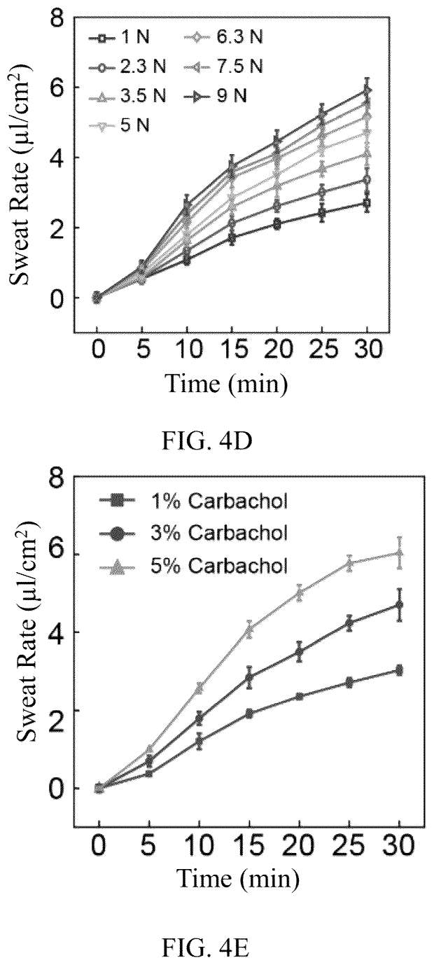

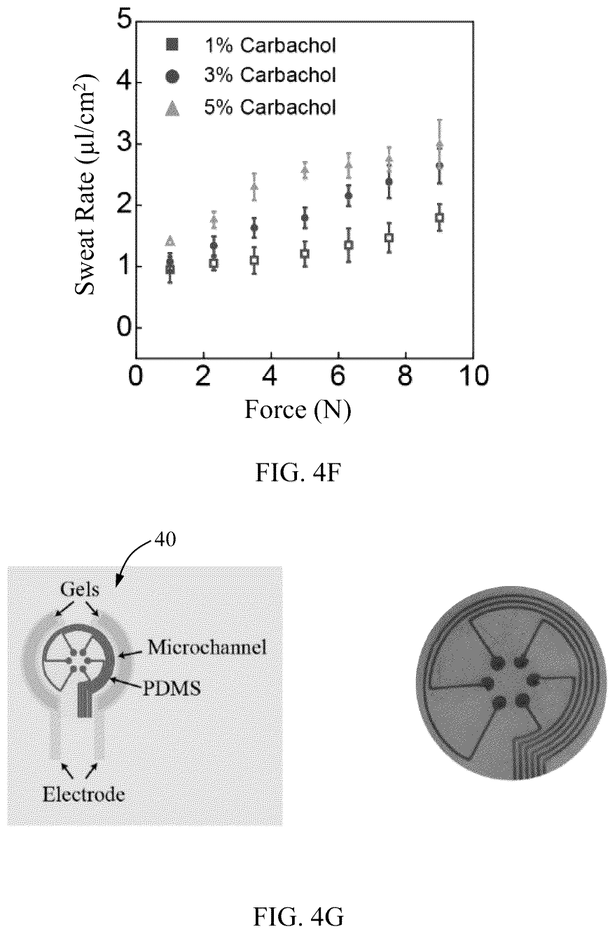

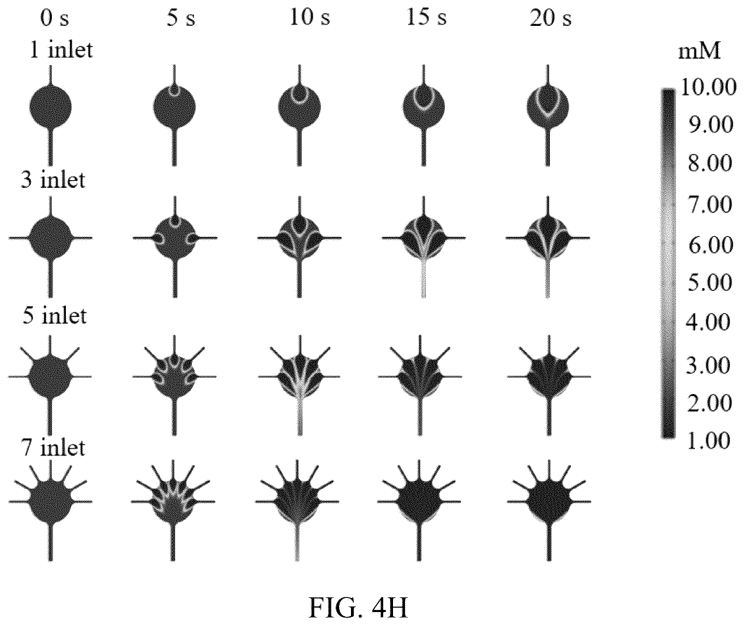

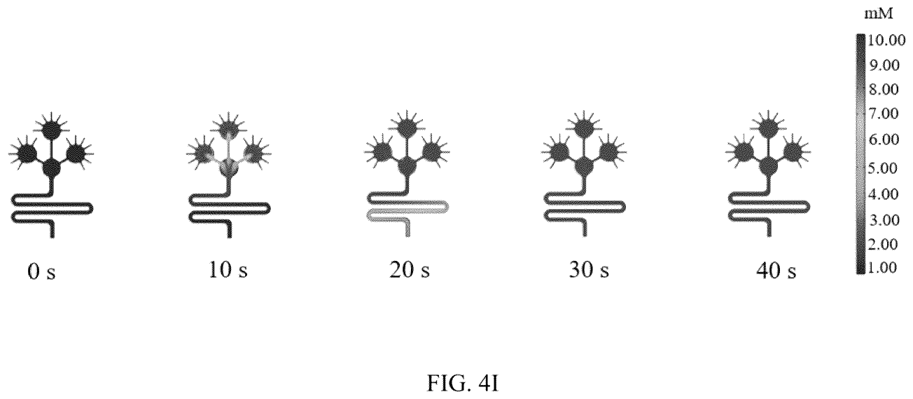

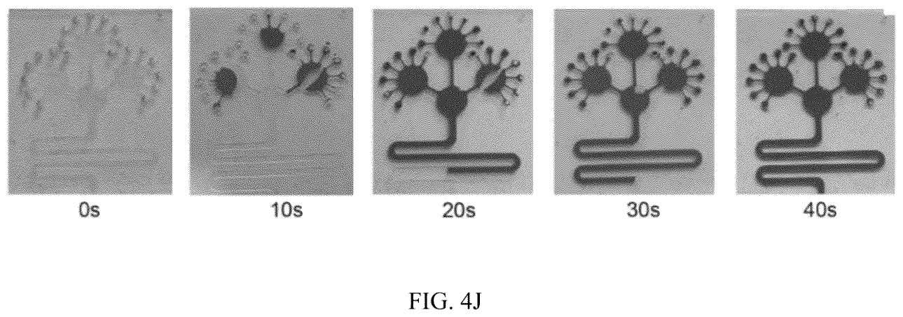

FIGS. 4A, 4B, and 4C illustrate the performance of a TENG under different pressures according to certain embodiments.

When it comes to the experimental studies as discussed herein, the TENG is manufactured in the way as described below. This is for illustrative purpose only. It will be appreciated that the TENG can be made in other ways according to practical scenarios. In some embodiments, the system, apparatus, device, etc. according to the present disclosure may incorporate various TENG. Turning back to the experimental studies, specifically, the manufacturing process of the layered TENG is achieved with a symmetrical structure, as illustrated in FIG. 3A. A 100 μm thick polyimide film is folded into six layers to serve as a substrate, with each layer having an area of 40 mm×40 mm. Copper electrodes are plated on both sides of the polyimide film using the flexible printed circuit board technology, with an area of 35 mm×35 mm for each copper electrode. A FEP film is then alternatively attached to one of the copper electrodes on the same side of the polyimide film, thereby forming a pair of electrodes. The FEP film has a thickness of 25 μm. The bare copper electrodes (i.e., those being not coated with the FEP film) serve as an output electrode, and together with the FEP-coated electrode, constitute the output end of the TENG.

The voltage and current outputs of the TENG are measured at pressures ranging from 1 N to 9 N. As can be seen, both voltage and current increase with the pressure increase with a maximum power output of 240 V and 17 μA, respectively. Such high power density (1.7-3.2 mW, see FIG. 4C) of the TENG can be achieved under light tapping at a frequency of around 2 Hz, which offers great practical application possibility as 2 Hz tapping can be easily achieved by users.

The effect of the formulation of compounds loaded in the iontophoresis process on the sweat extraction is also studied. One or more embodiments show the modulation of the sweat rate over the variations of the compounds. FIG. 4G illustrates a device 40 designed to measure the rate of sweat flow, where the gels are hydrogel. The device 40 comprises electrodes that contains carbachol, used to apply an electric field, and features an inner circular microfluidic channel design with six channels. To improve sweat visualization, coloured dye is added at the inlet of the microfluidic channel during testing. Sweat's flow rate is then calculated by multiplying the total length of the coloured liquid in the microfluidic channel by the cross-sectional area of the microfluidic channel and dividing it by time. The results show that the sweating volume directly relates to the loaded force, where the loading force from 1 to 9 N induces a sweat volume from 2.7 to 5.9 μl of sweat per square centimetre within 30 minutes (FIG. 4D). Furthermore, different concentrations of carbachol (1%, 3%, 5%) are tested and compared to study their sweat extraction rate differences, with a tapping force of 5 N and tapping frequency of 2 Hz. High concentrations of carbachol are found to be more effective in stimulating sweat secretion (FIG. 4E). The sweat extraction rates at different pressures and carbachol concentrations after ten minutes are found to be approximately 1-3 μl/cm² (FIG. 4F). These findings suggest that the feasibility of the TENG-based stimulation for sweat activation/extraction.

To improve or maximize the efficiency of low-volume sweat analysis and improve the temporal resolution of sensing, a compact and flexible microfluidic module is designed to isolate sweat sampling areas from iontophoresis gels. Numerical simulations are performed to optimize the geometric design of the microfluidic module, including number of inlets, orientation, and flow direction with respect to the reservoir geometry (FIG. 4H). As illustrated in FIG. 4H, the results indicate that the entire channel can be filled in just 5 seconds when the number of inlets is increased to seven. Based on these findings, the microfluidic system with seven inlets is used for the experiment. With an optimized design for inducing and sampling sweat, the multi-inlet microfluidic system allows for quick and convenient local induction of sweat, as well as rapid sampling over short period of time. The microfluidic system enables efficient collection of sweat samples for analysis and testing purposes. FIG. 4I shows a simulated sweat filling into the microfluidic channel at each inlet flow rate of 1.5 μL/min. According to the simulation results, it only takes about 40 seconds for the sweat to fully fill the microfluidic channel. FIG. 4J shows the feasibility of collecting sweat by injecting coloured liquid under the test, which is consistent with the simulation results.

The TENG can effectively generate stimulation to skin for sweat extraction. However, the present inventors have recognized that the transient pulse nature from the power generated by the TENG also brings the hurdles of continuous power supply for electronics. Therefore, one or more SABs are introduced in the system. As a result, the TENG serves as the ignition while the SABs serve as a power storage. Therefore, the TENG triggered SAB powering module can stably provide power output to various electronics within the SEMS.

According to one or more embodiments, the SABs are manufactured this way. The initial stage of producing SABs involves cutting an absorbent paper and a Mg sheet into a specified size of 1 cm×1 cm. The next step is to submerge the absorbent paper in an aqueous solution containing graphene that is uniformly dispersed, followed by drying at a temperature of 100° C. for 10 minutes. This process ensures that the graphene is evenly spread on the surface of the absorbent paper. After that, the graphene-coated paper along with the Mg sheet is placed adjacent to each other on the air-permeable material. Lastly, a thin layer of cotton that contains potassium chloride (KCl) powders is attached to the electrodes to create the SABs. It will be appreciated that this is merely for purpose of illustrating and acquiring experimental results for demonstration. In one or more embodiments, the SABs may be fabricated in other ways. Further, to improve or optimising the performance of the SABs, various variations may be designed and tested.

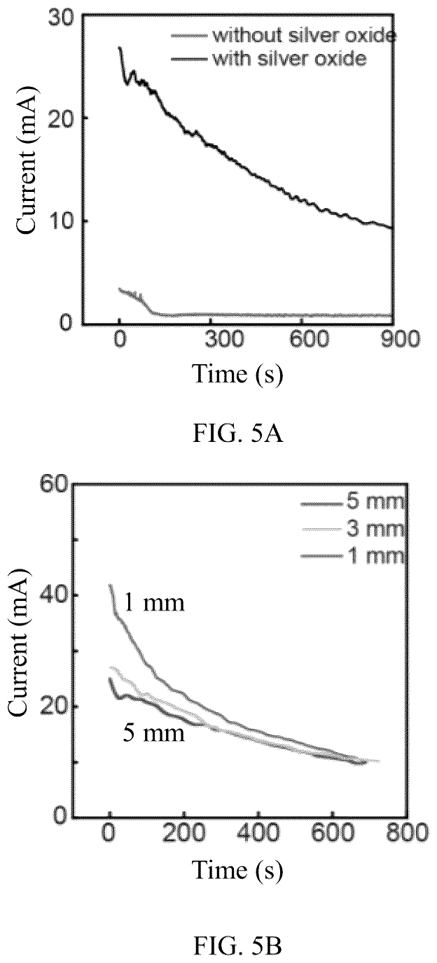

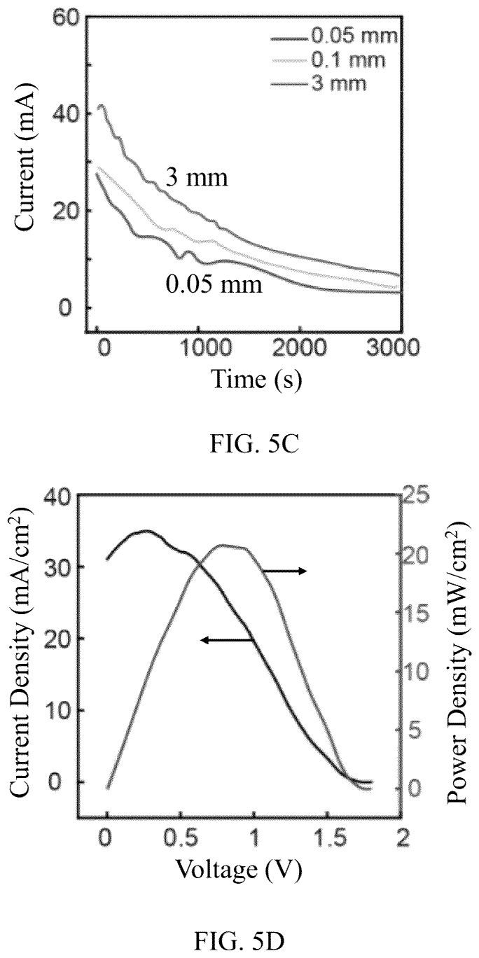

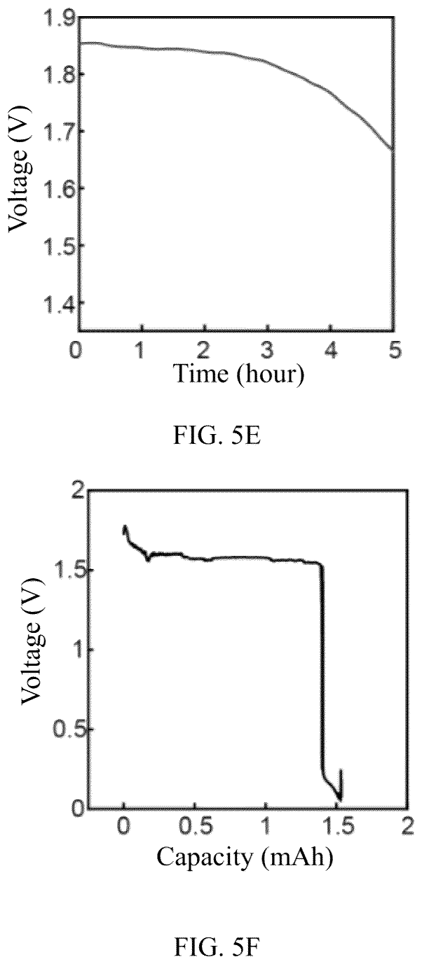

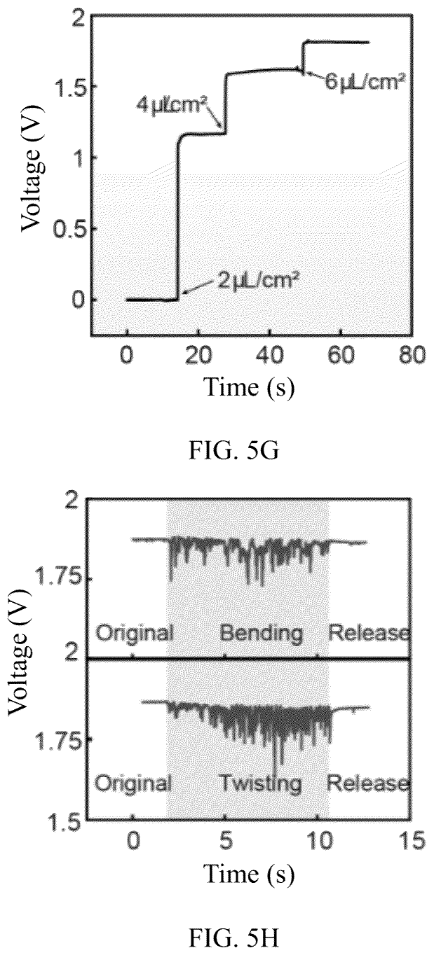

Referring to FIGS. 5A-5I, according to one or more embodiments, silver oxide (Ag₂O)-coated carbon cloth serves as the cathode of the SAB for catalysing the oxygen reduction, while the Mg foil with a size of 10 mm×10 mm serves as the anode. It is observed by the present inventors that addition of Ag₂O to the cathode can significantly increase the current by over eight times (FIG. 5A). This is attributed to the excellent catalytic performance of Ag₂O for the oxygen reduction reaction. Moreover, it is observed by the present inventors that the distance between the cathode and anode, and the thickness of the anode Mg foil, etc. can also influence the device size, operation time, and output performance, etc. As shown in FIG. 5B, a shorter distance between the anode and cathode increases the short-circuit current of the SAB due to the reduced impedances. As shown in FIG. 5C, a thicker Mg foil increases the short-circuit current of the SAB. At a constant Mg foil thickness of 100 μm and ambient temperature of 25° C., the working duration of the SAB and the corresponding average short-circuit current drops from 3.52 h and 14.98 mA as the distance between electrodes increases from 0.5 to 2.5 cm. This result owns to the increase of the inner impedance of the SAB. In order to balance the lifetime and the flexibility of the SAB, 50 μm Mg foil is found to be the optimized value for the anode. Such SAB can exhibit a maximum current density of 33 mA/cm² and maximum power density of 24 mW/cm², and therefore is sufficient to power the electronics (FIG. 5D). FIG. 5E shows the open-circuit voltage of a representative SAB cell as a function of time after absorbing sweat. The voltage is found to stabilize at 1.66 V-1.86 V for more than 5 h. Furthermore, the discharge capacity of the SAB reaches 0.5 mA h in such a small cell (1 cm×2 cm) as its output voltage drops from 1.74 V to 0 V (FIG. 5F). To evaluate the effect of the absorbed sweat volume on the SAB's electrical performance, the open-circuit voltage of the SAB is measured by increasing artificial sweat volume gradually from 0 to 6 μL/cm² (FIG. 5G). The results show that the SAB can be easily activated by a very small amount of sweat volume of 2 μL/cm², and the corresponding open-circuit voltage of 1.2 V. In addition, The SAB exhibits great softness and stable performance under various mechanical deformations. The soft materials and simple device structures allow the SAB to maintain a stable output voltage of approximately 1.85 V even after being bent and twisted at least 50 times (FIG. 5H). In the dynamic mechanical test, the SAB's output voltage drops slightly from 1.74 V to 1.52 V under 180 degree folding in the middle (FIG. 5I), while the device can recover to its initial electrical performance after releasing. Similarly, the twisting test results also demonstrate the excellent flexibility and robustness of the SAB, as only small and recoverable jitters appear in the voltage during the twisting process.

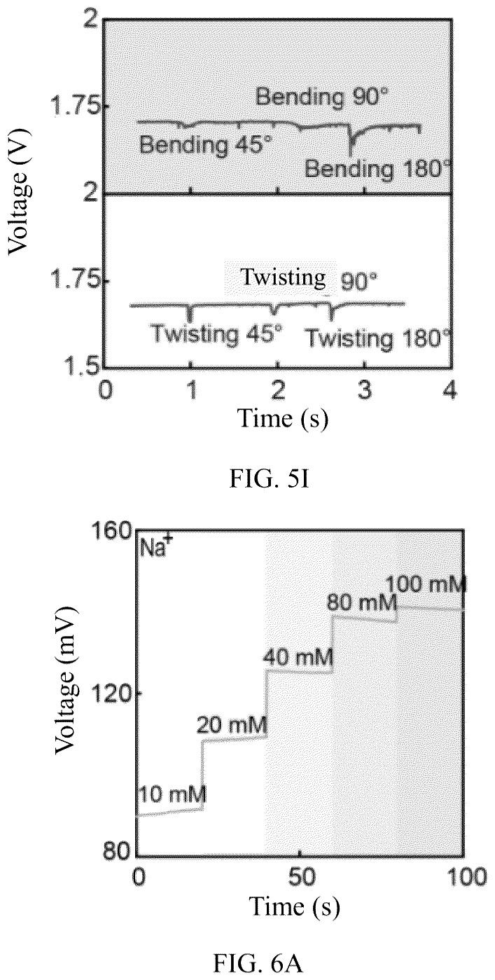

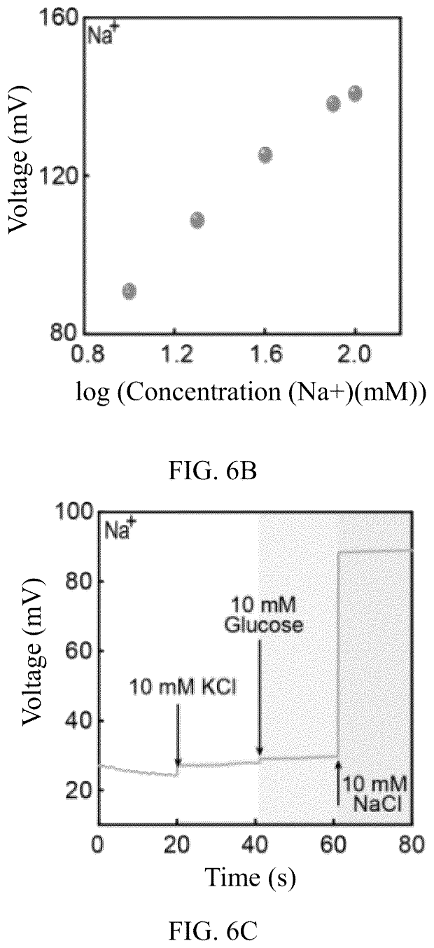

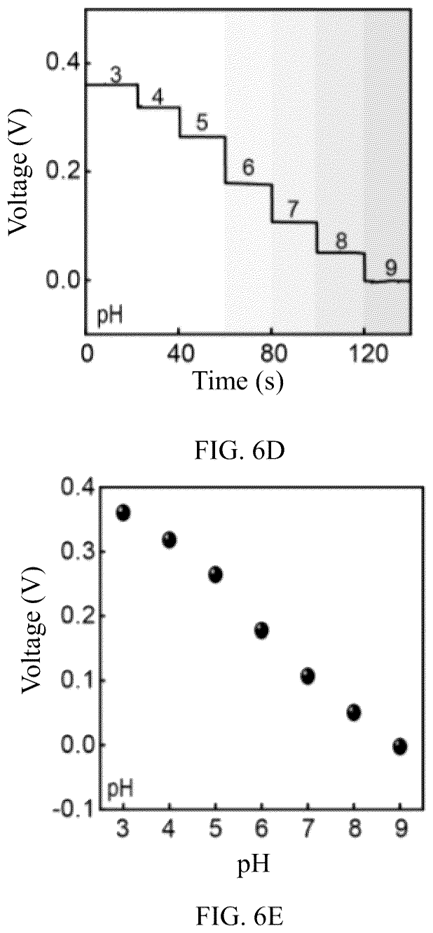

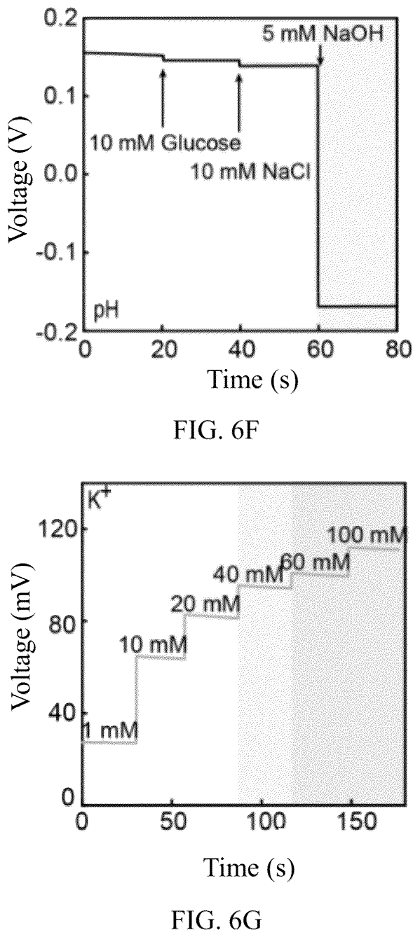

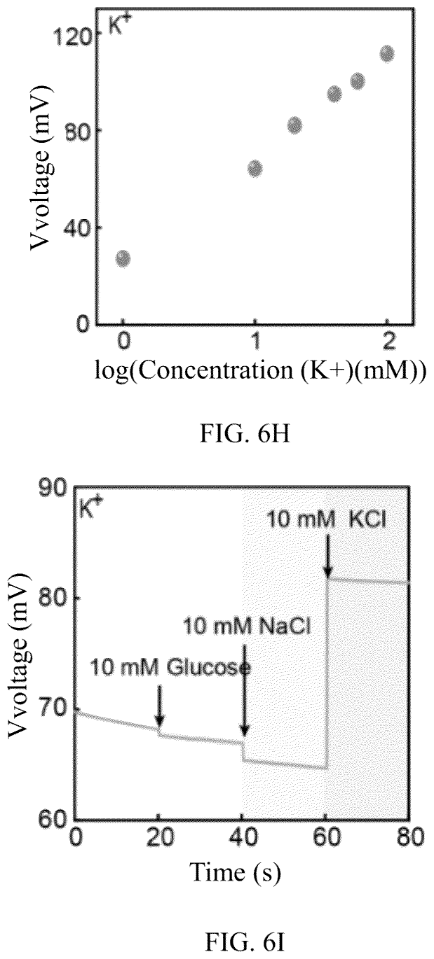

FIGS. 6A-6I show the characterizations of the flexible integrated sensing patch, which can real-time monitor the Na+ and K+ concentrations, as well as pH values in human sweat, thereby reflecting the health status of subjects under test.

In the present embodiments, the flexible integrated sensing patch is manufactured in this way. The fabrication of the sensor patch starts on the FPCB electrodes. For Na+ and K+ sensors, a layer of PEDOT: PSS is firstly electrodeposited on the gold electrode under a constant current of 141.36 μA for 1200 s. The electrolyte of electrodeposition is the mixed solution of 0.01 M 3,4-Ethylenedioxythiophene (EDOT) and 0.1 M polystyrene sulfonate (NaPSS). After that, 2 μL corresponding ionophore cocktail is dropped on the electrode and stored in refrigerator overnight. The Na+ ionophore cocktail includes 1 mg Na ionophore X. 0.55 mg sodium tetrakis [3,5-bis(trifluoromethyl)phenyl]borate (Na-TFPB), 33 mg polyvinyl chloride (PVC, K-value 72-1), and 65.45 mg bis(2-ethylehexyl) sebacate (DOS) dissolved in 610 μL of tetrahydrofuran 17. The difference between Na+ ionphore cocktail and K+ ionphore cocktail is using valinomycin to replace Na ionophore X as the ionophore. For the pH sensor, the layer of PANI is obtained on gold electrode by electrodeposition using cyclic voltammetry methods for 40 segments (−0.2 to 1 V at a scan rate of 0.1 Vs⁻¹). The electrolyte includes 0.1 M aniline and 1 M sulfuric acid. Besides, silver (Ag)/silver chloride (AgCl) ink is screen-printed on gold electrode to be the references. 2 μL of polyvinyl butyral (PVB) cocktail (79 mg PVB, 50 mg NaCl and 2 mg block polymer PEO-PPO-PEO dissolved in 1 mL methanol) is lastly dropped on the Ag/AgCl electrode to decrease or minimize the potential drift.

As shown in FIGS. 6A and 6B, the voltage output of Na+ sensor increases with the increase of Na+ concentration in the electrolyte. There is a linear relationship between the voltage values and the Na+ concentration. Further, the great selectivity of ionophore allows the sensor to exhibit great anti-interference ability (FIG. 6C). There are no obvious output responses when adding KCl and glucose into the electrolyte. In turn, only the analyte (NaCl) can cause significant change of the sensing signal. FIGS. 6D and 6E show the linear relationship between voltage outputs of the PANI-based pH sensor in a large range of 3 to 9. With the decrease of the hydrogen ion (H+) concentration in the electrolyte, the pH value increases and the conductivity of the PANI decreases, leading to lowered voltage outputs. It is worth to mention that the voltage fluctuation caused by interferents is negligible (FIG. 6F). Similar with the Na+ sensor, the K+ sensor also associates with PEDOT: PSS and corresponding ionphore film. As shown in FIGS. 6G and 6H, the outputs of the K+ sensor are linearly related to the K+ concentrations in a range of 1 mM to 100 mM with good anti-interference ability and selectivity (FIG. 6I). Overall, these sensors formed on the integrated flexible patch all exhibit excellent linearity, selectivity, and sensitivity, which can largely increase the detection accuracy for human sweat.

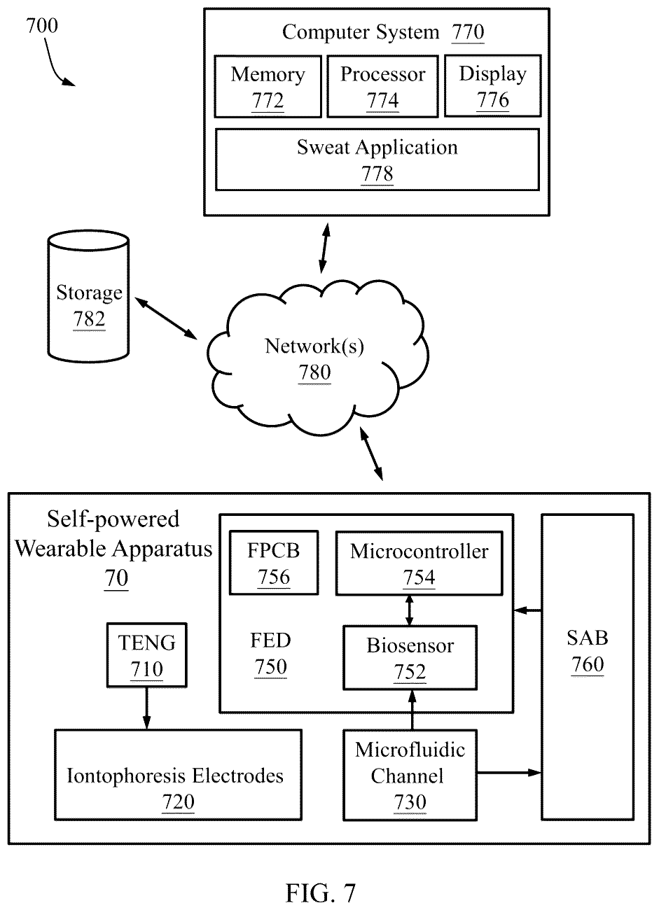

FIG. 7 illustrates a self-powered wearable system 700 according to certain embodiments. The system 700 comprises a self-powered wearable apparatus 70 and a computer system 770 that communicates with the apparatus 70 via one or more networks 780.

The apparatus 70 can be one or more specific implementations of the SEMS 200 as described above. The apparatus 70 includes a TENG 710, iontophoresis electrodes 720, at least one microfluidic channel 730, a FED 750 and at least one SAB 760. The FED 750 includes a FPCB 756, at least one biosensor 752 and a microcontroller 754.

The computer system 770 includes one or more computers or electronic devices, such as one or more servers, that include one or more components of computer readable medium (CRM) or memory 772, a processor or processing unit 774 (such as one or more processors, microprocessors, and/or microcontrollers), one or more displays 776, and a sweat application 778.

The memory 772 stores data (such as the sensed data by the biosensor 752) received from the apparatus 70. The memory 772 may store instructions that when executed cause the processor 774 to process the received data from the apparatus 70. The processed results may also be stored in the memory 772. The sweat application 778 may be an example of specialized hardware and/or software that assist in improving performance of a computer and/or execution of data processing discussed herein. The display 776 may provide a user interface such that users can interact with the apparatus 70 via the sweat application 778. For example, users may view on the display 776 the results or status in relation to one or more aspects of the health conditions for a person. Users may input instructions for remotely controlling operation of one or more aspects of the apparatus 70. For example, users may instruct which biosensor or biosensors are expected to operate. Users may instruct whether a real-time sensing and monitoring is desired, or which time period the sweat sensing is desired to operate.

The storage 782 may include one or more of memory or databases that store one or more of texts, image files, audio files, video files, software applications, and other information discussed herein. By way of example, the storage 782 may store texts, graphs, instructions or software application that can be retrieved by the computer system 770 over the network 780. For example, the storage 782 may store the historical record of the sensed data in relation to the health conditions of a person or a group of people. The computer system 770 may retrieve this historical record and generate comparison data between the past and the present for a particular person, or comparison data for a large number of people, or health report, etc.

The network 780 may include one or more of a cellular network, a public switch telephone network, the Internet, a local area network (LAN), a wide area network (WAN), a metropolitan area network (MAN), a personal area network (PAN), home area network (HAM), and other public and/or private networks. Additionally, the electronic devices need not communicate with each other through a network. As one example, electronic devices can couple together via one or more wires, such as a direct wired-connection. As another example, electronic devices can communicate directly through a wireless protocol, such as Bluetooth, NFC, UWB, Zigbee, and WiFi, or other wireless communication protocol.

As the self-powered wearable system is a TENG-based system, in one or more embodiments, it is also called a wearable TENG. The wearable TENG may be a versatile device or apparatus that can take various forms such as textile, insole, wristband, hand, and arm pressing, and convert biomechanical energy into electrical energy.

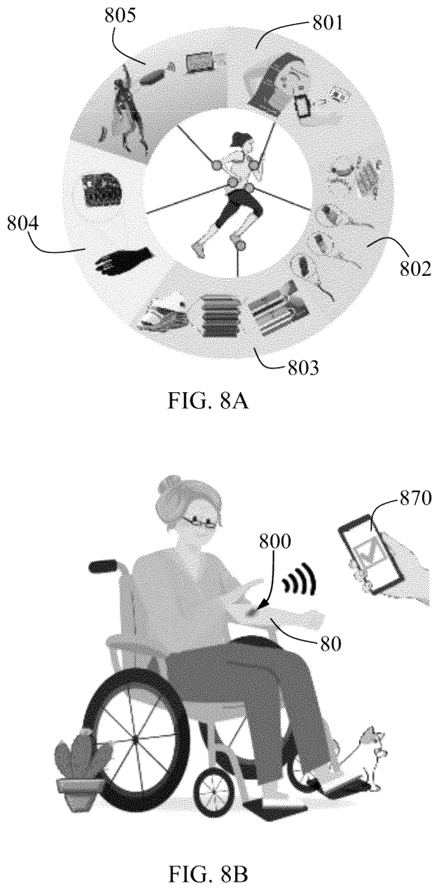

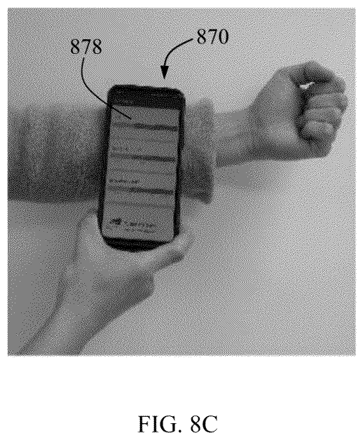

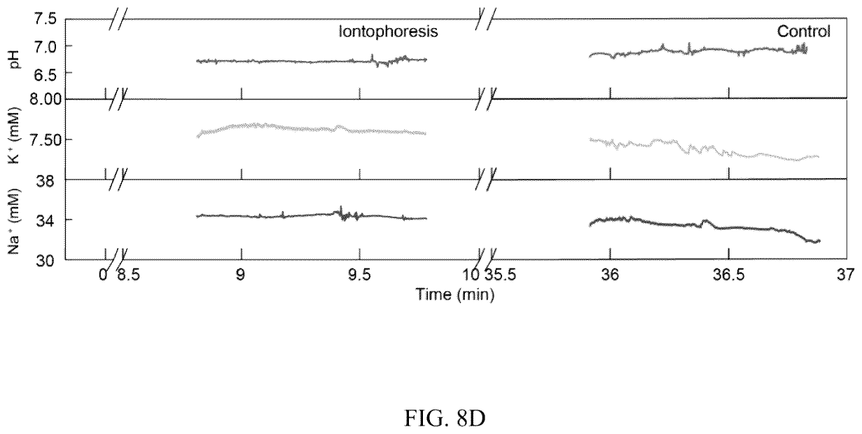

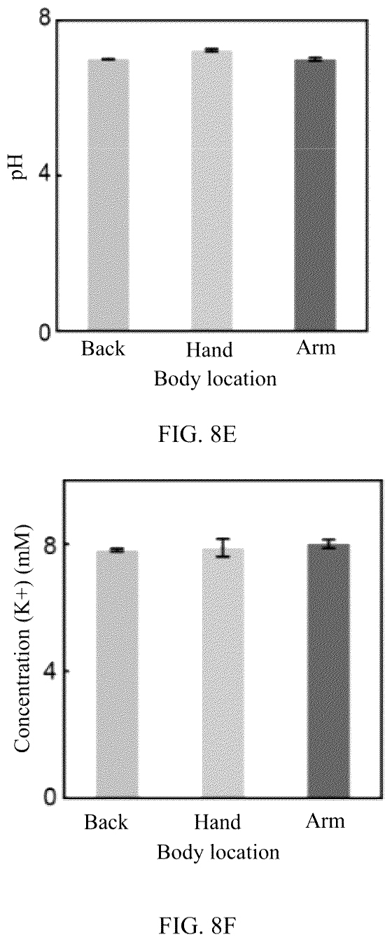

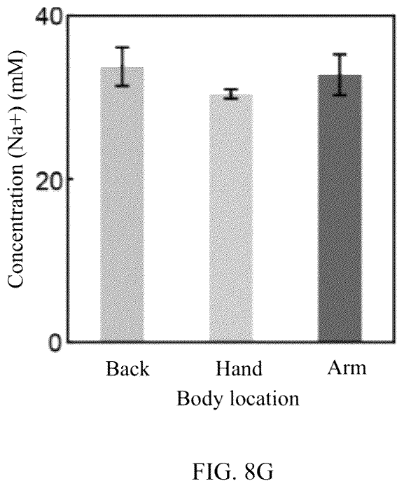

Similarly, as the SEMS as described above according to one or more embodiments is a TENG-based system, it may be called a TENG device in some embodiments. FIG. 8A shows some examples how the TENG device may function, such as by armpit pressing 801, clothes friction 802, sole pressing 803, hand motion 804, and joint bending 805. It is particularly useful for individuals who have difficulty in sweating through intense exercises, such as elderly people or those with physical disabilities. As illustrated in FIG. 8B, tapping the TENG device 800 on a user's arm 80 allows to collect TENG energy through localized movements, which can accelerate sweating and help obtain sweat information quickly. The obtained information can be sent to a smartphone 870 for viewing or processing.

FIG. 8C shows a real-life example of a TENG device placed on the arm of a subject for self-powered sweat collection and SAB triggering. In this example, the sensors are powered by the SAB and the data are wirelessly transmitted by NFC to the smartphone 870 having a sweat application 878 displayed on the user interface. Specifically, the data collected by the sensors are converted into digital signals that are stored in a memory via Inter-Integrated Circuit (I2C), and can be transmitted to a mobile terminal through the NFC chip. The GUI in the smartphone 870 can display and store the received data for further analysis.

FIG. 8D illustrates the impact of the TENG stimulation on the pH, Na+ concentration and K+ concentration of sweat. The results demonstrate that the TENG stimulation enables detection of sweat information in approximately 8.7 minutes. In contrast, the control group without electrical stimulation requires around 35.9 minutes when they are at rest. This suggests that the TENG stimulation significantly accelerates the response time for sweat detection.

FIGS. 8E and 8F demonstrate consistency of the results collected by the biosensors from different body locations. Overall, the self-sweat collection and self-powered device offers a convenient and effective solution for healthcare monitoring in wearable systems.

It will be appreciated that herein layered TENG device is used as a power source to demonstrate the effectiveness of the TENG device. In practical applications, the TENG device can take various forms or variations to provide energy for electrically stimulated sweating for human skins.

As used herein, the term “monitoring” refers broadly to obtaining one or more parameters in relation to an object (such as human skin, a human's health conditions, etc) by measuring, detecting, sensing, or the like, or by analyzing data, parameters, etc. The term “monitoring” may refer to a continuous process, or a discontinued activity, or a one-off activity.

As used herein, the term “wearable” refers broadly to being attachable to a human body in certain way so as to be physically portable.

It will further be appreciated that any of the features in the above embodiments of the invention may be combined together and are not necessarily applied in isolation from each other. Similar combinations of two or more features from the above-described embodiments or preferred forms of the invention can be readily made by one skilled in the art.

Unless otherwise defined, the technical and scientific terms used herein have the plain meanings as commonly understood by those skill in the art to which the example embodiments pertain. Embodiments are illustrated in non-limiting examples. Based on the above disclosed embodiments, various modifications that can be conceived of by those skilled in the art would fall within spirits of the example embodiments.

What is claimed is:

1. A sweat extraction and monitoring system (SEMS), comprising:
a triboelectric nanogenerator (TENG) for inducing localized sweating for a human skin to generate sweat;
iontophoresis electrodes electrically connected to the TENG and configured to be operably electrically contacting the human skin;
at least one microfluidic channel for receiving the sweat;
at least one biosensor for sensing the sweat received in the at least one microfluidic channel; and
at least one sweat-activated battery (SAB) configured to be actuatable by the sweat for powering the at least one biosensor.

2. The SEMS of claim 1, wherein the iontophoresis electrodes are loaded with carbachol.

3. The SEMS of claim 1, wherein the TENG has a stacked layer structure comprising:
a plurality of polyimide layers folded from a polyimide film;
copper electrodes formed on each of the plurality of polyimide layers; and
fluorinated ethylene propylene (FEP) configured to be alternatively attached onto the copper electrodes to form FEP-attached copper electrodes,
wherein the copper electrodes that are not attached with FEP and the FEP-attached copper electrodes constitute output terminals of the TENG.

4. The SEMS of claim 3, wherein the stacked layer structure further comprises a gold film formed onto the plurality of polyimide layers.

5. The SEMS of claim 1, further comprising a flexible polyimide substrate, the iontophoresis electrodes being formed on the flexible polyimide substrate.

6. The SEMS of claim 5, further comprising a hydrogel patch disposed on the iontophoresis electrodes such that the hydrogel patch operatively contacts the human skin.

7. The SEMS of claim 6, wherein the hydrogel patch comprises a first hydrogel patch and a second hydrogel patch, and wherein the iontophoresis electrodes comprise an anode and a cathode, the first hydrogel patch being loaded with sodium chloride (NaCl) and disposed on the anode, the second hydrogel patch being loaded with carbachol and disposed on the cathode.

8. The SEMS of claim 1, wherein the at least one biosensor comprises one or more sensors selected from a group consisting of a sodium ion (Na+) sensor, a potassium ion (K+) sensor, and a pH sensor.

9. The SEMS of claim 1, wherein the at least one biosensor comprises a sodium ion (Na+) sensor, the Na+ sensor comprising a layer of poly(3,4-ethylenedioxythiophene: poly(sodium 4-styrenesulfonate) (PEDOT: PSS) and a layer of ionophore disposed onto the layer of PEDOT: PSS and serving as a sensing area for sensing sodium ions.

10. The SEMS of claim 1, wherein the at least one biosensor comprises a potassium ion (K+) sensor, the potassium ion (K+) sensor comprising a layer of PEDOT: PSS and a layer of ionophore disposed onto the layer of PEDOT: PSS and serving as a sensing area for sensing potassium ions.

11. The SEMS of claim 1, wherein the at least one biosensor comprises a pH sensor comprising polyaniline.

12. The SEMS of claim 1, wherein the at least one SAB comprises a cathode and an anode, the cathode comprising a layer of silver oxide (Ag₂O)-coated carbon cloth.

13. The SEMS of claim 1, wherein the at least one SAB comprises a cathode and an anode, the anode comprising a magnesium foil.

14. The SEMS of claim 1, wherein the at least one microfluidic channel comprises a plurality of inlets for receiving the sweat.

15. A sweat extraction and monitoring system (SEMS), comprising:
a triboelectric nanogenerator (TENG) for inducing localized sweating for a human skin to generate sweat;
iontophoresis electrodes electrically connected to the TENG and configured to be operably electrically contacting the human skin;
at least one microfluidic channel for receiving the sweat;
a flexible electronic device comprising a flexible printed circuit board (FPCB), at least one biosensor disposed on the FPCB for sensing the sweat received in the at least one microfluidic channel to generate sensed data, and a microcontroller, the microcontroller being configured to transmit the sensed data to an external computer system; and
at least one sweat-activated battery (SAB) configured to be actuatable by the sweat for powering the at least one biosensor and the microcontroller.

16. The SEMS of claim 15, wherein the SEMS is encapsulated with polydimethylsiloxane (PDMS).

17. A self-powered wearable system, comprising:
a wearable sweat apparatus comprising:
a triboelectric nanogenerator (TENG) for inducing localized sweating for a human skin to generate sweat;
iontophoresis electrodes electrically connected to the TENG and configured to be operably electrically contacting the human skin;
at least one microfluidic channel for receiving the sweat;
a flexible electronic device comprising a flexible printed circuit board (FPCB), at least one biosensor disposed on the FPCB for sensing the sweat received in the at least one microfluidic channel to obtain sensed data, and a microcontroller electrically communicating with the at least one biosensor; and
at least one sweat-activated battery (SAB) configured to be actuatable by the sweat for powering the at least one biosensor and the microcontroller; and
a computer system for electrically communicating with the microcontroller for receiving the sensed data.

18. The self-powered wearable system of claim 17, wherein the sensed data comprises one or more of data selected from a group consisting of sodium ion (Na+) concentration, potassium ion (K+) concentration, and pH values of the sweat.

19. The self-powered wearable system of claim 17, wherein the microcontroller communicates with the computer system via a protocol selected from a group consisting of near field communication (NFC), Bluetooth, ultra-wide band (UWB), Zigbee, and WiFi.

20. The self-powered wearable system of claim 17, wherein the computer system comprises a user interface configured to allow a user to remotely operate the wearable sweat apparatus.
